(19) United States
(12) Patent Application Publication
(10) Pub. No.: US 2026/0026940 A1
(43) Pub. Date: Jan. 29, 2026

(54) SYSTEMS AND METHODS FOR TOTAL ANKLE ARTHROPLASTY

(71) Applicant: WRIGHT MEDICAL TECHNOLOGY, INC., Memphis, TN (US)

(72) Inventors: David R. TUTTLE, Memphis, TN (US); Sach COMBS, Millcreek, UT (US); Matthew John OBROCK, Maumee, OH (US); Ramon LUNA, Arlington, TN (US)

(73) Assignee: WRIGHT MEDICAL TECHNOLOGY, INC., Memphis, TN (US)

(21) Appl. No.: 19/241,507

(22) Filed: Jun. 18, 2025

Related U.S. Application Data
(60) Provisional application No. 63/674,805, filed on Jul. 24, 2024.

(51) Int. Cl. A61F 2/42; A61B 17/56; A61F 2/30

(52) U.S. Cl. CPC A61F 2/4202; A61B 17/56; A61B 2017/564; A61F 2002/30112; A61F 2002/30215; A61F 2002/30331; A61F 2002/30405; A61F 2002/30476; A61F 2002/30604; A61F 2002/4205; A61F 2002/4207

(57) ABSTRACT

An ankle arthroplasty implant system and method for installing into a resected ankle is provided including a multi-component tibia stem, a tray implant that includes side pockets extending into the tray implant. The tray implant further includes a proximal surface configured to engage the multi-component stem, and a distal side, including a recess. A talar dome implant is also provided that includes a convex proximal articular surface, an anterior side, a posterior. The ankle arthroplasty implant system further includes a polymer insert configured to be inserted in the tray implant recess.

### CROSS-REFERENCE TO RELATED APPLICATION

This application claims priority to U.S. Provisional Patent Application No. 63/674,805, filed on Jul. 24, 2024, the entirety of which is incorporated herein by reference.

### FIELD OF DISCLOSURE

The disclosed systems and methods relate to the field of orthopedic implants.

### BACKGROUND

A variety of talar dome implants are used in ankle joint prosthetic procedures. These may include flat cut, chamfered, and stemmed talar dome implants. A surgeon may select the appropriate type of talar dome implant during a procedure based on each patient's unique joint space or anatomical needs. However, fitting a particular selected talar dome implant into a surgical bone resection can be difficult, or require a larger resection than may be preferred for the patient.

During ankle joint prosthetic procedures, surgeons may insert a talar dome trial into the joint space to determine the correct size talar dome implant to use in the procedure, and to install pin holes in the talus to prepare the talar surface. The surgeon would subsequently insert the talar dome implant. Once a surgeon has inserted a talar dome implant into the joint space during an ankle joint prosthetic procedure, the surgeon would typically use a talar dome impactor to secure the talar dome implant to drilled holes in the talus.

Many total ankle replacement prosthesis systems include an assembly including a tibia tray and a bearing component. Generally, the tibia tray is installed on to the distal end of a patient's tibia that has been prepared to receive the tibia tray. In some cases, the tibia surface is prepared in a similar manner to the talar surface, allowing for installation using pegs as fixation. However, if the tibia bone is weakened or deteriorated, a longer-stemmed tibia tray implant can be inserted into an intramedullary path in the tibia to increase the surface area between the implant anchor and the soft, spongy bone marrow. The bone marrow will grow and coalesce around the anchor, and the stemmed tibia tray implant will have improved surface area and leverage against the tibia, supporting ankle momentum. However, the task of installing the long-stemmed tibia tray is difficult when fitting into a relatively planar surgical resection for installation. Further, even if the long-stemmed tibia tray implant were largely planar, it must be pivoted through the surgical opening, and into the intramedullary path, which is generally perpendicular to the surgical resection. Thus, a long-stemmed tibia tray capable of both fitting through a planar opening, as well as entering a space perpendicular to the planar opening without requiring a sweeping, arcing intramedullary path, is desired.

After the tibia tray implant is installed, the bearing component is often removably engaged to the bottom or distal surface of the tibia tray by a sliding mechanism. Despite the sliding and removably-engaged nature of the relationship between the tibia tray and the bearing component, in practice, once seated the bearing component should be unable to slide out from the tibia tray, unless urged to do so in a later surgery to replace the bearing component. Thus, a bearing component and tibia tray system that utilizes an improved locking seating scheme or configuration is desired. Additionally, post-installation, the tibia tray, bearing component, and talar dome implant should be in tight contact, to function as a talocrural joint. Thus, a bearing component capable of fitting through as short a planar opening as possible, to maximize the post-operative tautness of the talocrural joint, is desired.

### SUMMARY

An ankle arthroplasty implant system is provided including a multi-component tibia stem configured for engaging a distal end of a tibia in a patient's ankle joint and including a first component stem and a second component stem. The ankle arthroplasty implant system further includes a tray implant, including an anterior side. The anterior side includes a slot extending into the tray implant, with the slot being generally perpendicular to the anterior side. The tray implant also includes one or more side pockets located on the medial and/or lateral sides of the slot, extending into the tray implant and being generally parallel to the slot. The tray implant further includes a proximal surface configured to engage the multi-component stem, and a distal side, including a recess. The ankle arthroplasty implant system further includes a talar dome implant, the talar dome implant including a convex proximal articular surface, an anterior side, a posterior side, and a bone-contacting distal surface opposite from the convex proximal articular surface. The bone-contacting distal surface is a concave surface. The bone-contacting distal surface is configured to abut against a resected surface of the talus of the ankle joint, where the resected surface has a shape that is complementary to the bone-contacting distal surface. The ankle arthroplasty implant system further includes a polymer insert, configured to be inserted in the tray implant recess. The polymer insert includes an anterior face, including one or more blind holes, and a concave distal surface configured to substantially conform to and engage with the proximal articular surface of the talar dome implant.

Also disclosed is a method that provides forming an intramedullary path in a patient's tibia at the distal end of the tibia by drilling through the associated calcaneus bone. The method includes forming an ankle joint space by resecting the distal end of the patient's tibia and the proximal end of the patient's talar bone, such that the intramedullary path in the tibia presents an opening into the ankle joint space. The method further includes forming a convex cut to the resected proximal end of the patient's talar bone. The method further includes reaming the intramedullary path to a desired diameter and depth. The method still further includes inserting a first component stem of a multi-component stem into the ankle joint space and into the intramedullary path. The method further includes inserting a second component stem of the multi-component stem into the ankle joint space and coupling it to the first component stem, thus, forming an assembled stem. Additional component stems of the multi-component stem may be coupled to the assembled stem, extending the assembled stem. The assembled stem is advanced into the intramedullary path. The method yet further includes inserting a tray implant into the ankle joint space and coupling the tray to the assembled stem. Additionally, the method includes affixing a talar dome implant to the chamfered cut proximal end of the talar bone. Further, the method includes inserting a polymer insert into a tray implant recess on the distal side of the tray implant.

A polymer insert implant with an improved posterior face for inserting the polymer insert implant into a tibia tray mounted at the distal end of a tibia is also disclosed.

Also disclosed is an improved polymer insert implant and tibia tray for improved locking and resistance after the polymer insert is inserted into the tibia tray mounted at the distal end of a tibia.

### BRIEF DESCRIPTION OF THE DRAWINGS

The figures provided with this disclosure are schematic and are not necessarily to scale. The figures are not intended to show the actual dimensions or actual relative dimensions unless otherwise specified.

FIG. 1 shows a total ankle arthroplasty implant according to an embodiment of the present disclosure.

FIG. 2A shows a top stem of the multi-component stem of the total ankle arthroplasty implant according to an embodiment of the present disclosure.

FIG. 2B shows a mid stem of the multi-component stem of the total ankle arthroplasty implant according to an embodiment of the present disclosure.

FIG. 2C shows a base stem of the multi-component stem of the total ankle arthroplasty implant according to an embodiment of the present disclosure.

FIG. 2D shows a top view of the top stem of the multi-component stem of the total ankle arthroplasty implant according to an embodiment of the present disclosure.

FIG. 2E shows a side view of the top stem of the multi-component stem of the total ankle arthroplasty implant according to an embodiment of the present disclosure.

FIG. 2F shows an isometric view of the top stem of the multi-component stem of the total ankle arthroplasty implant according to an embodiment of the present disclosure.

FIG. 2G shows a bottom view of the top stem of the multi-component stem of the total ankle arthroplasty implant according to an embodiment of the present disclosure.

FIG. 2H shows a top view of the mid stem of the multi-component stem of the total ankle arthroplasty implant according to an embodiment of the present disclosure.

FIG. 2I shows a side view of the mid stem of the multi-component stem of the total ankle arthroplasty implant according to an embodiment of the present disclosure.

FIG. 2J shows an isometric view of the mid stem of the multi-component stem of the total ankle arthroplasty implant according to an embodiment of the present disclosure.

FIG. 2K shows a bottom view of the mid stem of the multi-component stem of the total ankle arthroplasty implant according to an embodiment of the present disclosure.

FIG. 2L shows a top view of the base stem of the multi-component stem of the total ankle arthroplasty implant according to an embodiment of the present disclosure.

FIG. 2M shows a first side view of the base stem of the multi-component stem of the total ankle arthroplasty implant according to an embodiment of the present disclosure.

FIG. 2N shows a second side view of the base stem of the multi-component stem of the total ankle arthroplasty implant according to an embodiment of the present disclosure.

FIG. 2O shows an isometric view of the base stem of the multi-component stem of the total ankle arthroplasty implant according to an embodiment of the present disclosure.

FIG. 2P shows a bottom view of the base stem of the multi-component stem of the total ankle arthroplasty implant according to an embodiment of the present disclosure.

FIG. 3 shows a polymer insert of the total ankle arthroplasty implant according to an embodiment of the present disclosure.

FIG. 4 shows a talar dome implant with a chamfered profile according to an embodiment of the present disclosure.

FIG. 5A shows an anterior-side view of the multi-component stem and a tibia tray coupled together according to an embodiment of the present disclosure.

FIG. 5B shows a section view of the tibia tray shown in FIG. 5A, where the section is taken through the slot 141 of the tibia tray along a plane that is parallel to the proximal surface 145.

FIG. 6A shows an isometric view of a polymer insert of the total ankle arthroplasty implant according to an embodiment of the present disclosure.

FIG. 6B shows a distal-side view of a polymer insert of the total ankle arthroplasty implant according to an embodiment of the present disclosure.

FIG. 6C shows an inferior-side view of a polymer insert of the total ankle arthroplasty implant according to an embodiment of the present disclosure.

FIG. 7A shows a side view of the total ankle arthroplasty implant during insertion of the polymer insert according to an embodiment of the present disclosure.

FIG. 7B shows a detail isometric section view of the total ankle arthroplasty implant during insertion of the polymer insert according to an embodiment of the present disclosure.

FIG. 8A shows a cross-section view of the total ankle arthroplasty implant assembly in which the polymer insert is inserted and seated into the tibia tray according to an embodiment of the present disclosure.

FIG. 8B shows a detail area diagram of the locking mechanism shown in FIG. 8A

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures. In this detailed description and the following claims, the words proximal, distal, anterior or plantar, posterior or dorsal, medial, lateral, superior, and inferior are defined by their standard usage for indicating a particular part or portion of a bone, instrument, or apparatus according to the relative disposition of the natural bone or directional terms of reference. For example, “proximal” means the portion of an instrument or implant or implant nearest the torso, while “distal” indicates the portion of the instrument or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

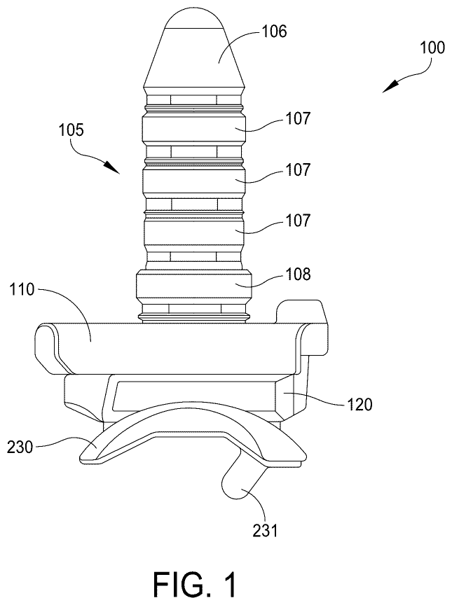

Referring to FIGS. 1 through 5B, a total ankle arthroplasty system according to the present disclosure will now be described. FIG. 1 shows a full assembly of a total ankle arthroplasty implant system 100 according to an embodiment of the present disclosure. An exemplar total ankle arthroplasty implant 100 can include a talar dome implant 230, a tibia tray implant 110, and a multi-component stem 105. For the purposes of discussion herein, the components of the total ankle arthroplasty implant 100 will be described in the context of a left ankle arthroplasty. Therefore, the anatomical directions anterior, posterior, medial, and lateral used in describing the various portions of the components of the implant 100 refer to the corresponding anatomical directions in the context of the left ankle of a patient.

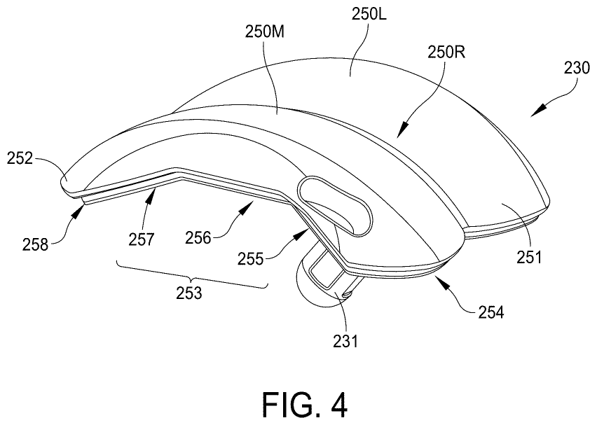

Some detailed structures of a talar dome implant 230 are shown in FIG. 4. Generally, the talar dome implant 230 is affixed to a talus bone, which has been resected in a complementary manner to accommodate the talar dome implant 230. The talar dome implant 230 is generally attached to the talus bone by one or more pegs 231, which are driven into the talus bone. The talus bone may be pre-drilled to accommodate the pegs 231 of the talar dome implant 230. The talar dome implant 230 is configured to act as one part of a replacement ankle joint in a patient.

On the tibia side of the replacement ankle joint, the total ankle arthroplasty implant 100 includes a tibia tray implant 110. The tibia tray implant 110 is affixed to the distal end of the tibia, which may be resected in a complementary manner to accommodate the tibia tray implant 110.

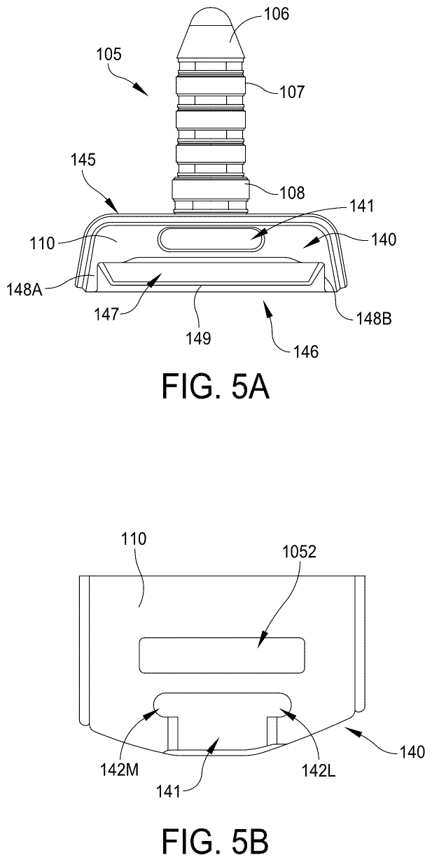

Referring to FIGS. 5A-5B, the tibia tray implant 110 comprises an anterior end 140 that includes a slot 141 extending into the tibia tray implant 110. The slot 141 is configured to receive an instrument for holding and handling the tibia tray implant 110. The tibia tray implant 110 further includes a proximal surface 145 configured to engage the multi-component stem 105, and a distal side 146, including a recess 147.

To facilitate the movement of the replacement ankle joint, the total ankle arthroplasty implant generally includes a polymer insert 120. The polymer insert 120 is configured to function like cartilage, and forms the connection between the tibia tray implant 110 and the talar dome implant 230. The polymer insert 120 in this embodiment slots into the tibia tray implant 110, and the polymer insert 120 then moves with the tibia tray implant 110 and articulates against the talar dome implant 230. In this example, the tibia tray 110 is affixed to the tibia by a multi-component modular stem component 105.

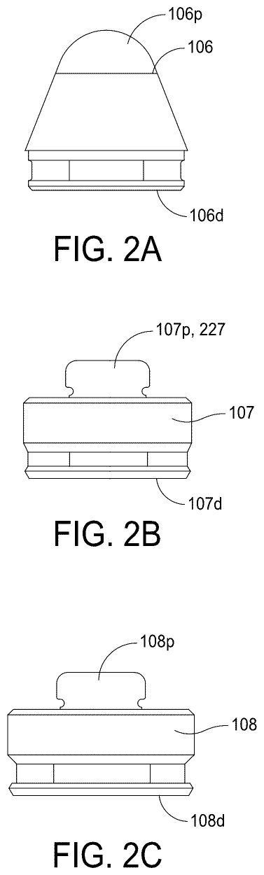

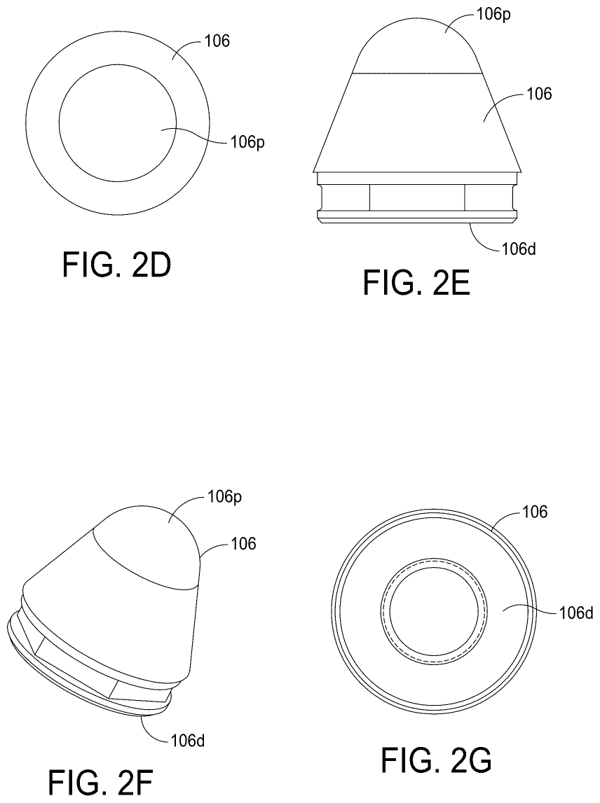

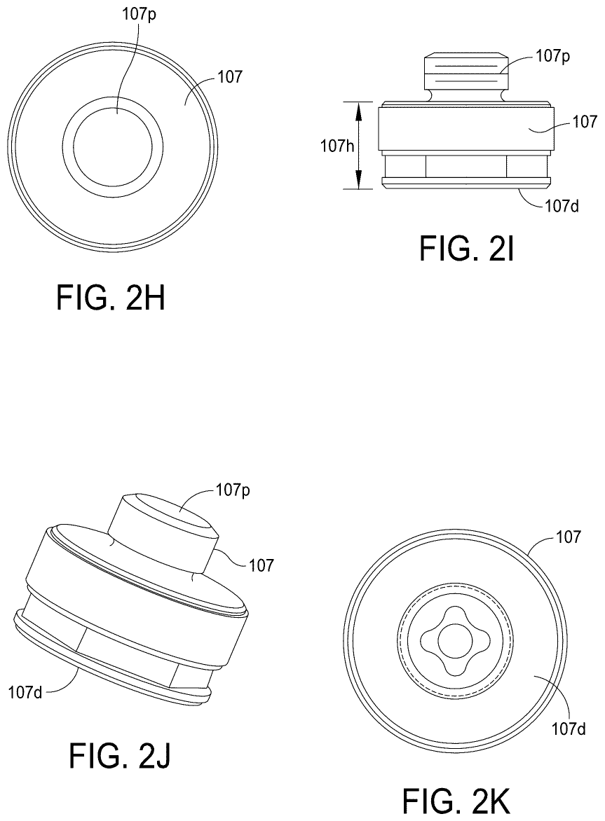

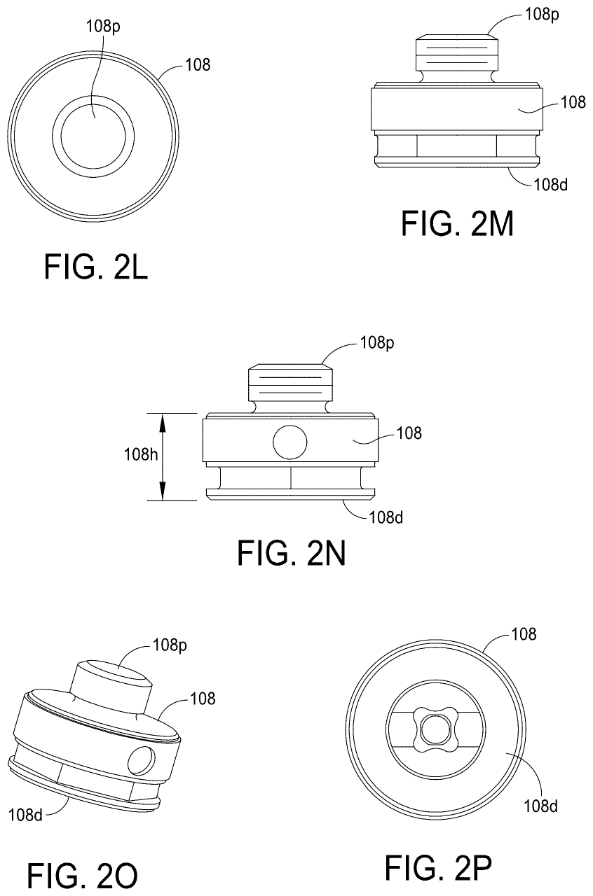

Referring to FIGS. 1-2P, the multi-component modular stem 105 can comprise a top stem 106 (maybe referred to as a first component stem), a base stem 108 (maybe referred to as a second component stem), and optional one or more mid stems 107 (may be referred to as a third component stem). In the example illustrated in FIG. 1, three mid stems 107 are employed in the total ankle implant system 100. The height of the multi-component stem 105 can be adjusted by selecting appropriate number of the mid stems 107. For example, from none to any number of mid stems 107 can reasonably be used to facilitate connectivity and support to the tibia depending on the needs of a particular patient. The modularity of the multi-component stem 105 allows a surgeon to assemble the multi-component stem 105 with an appropriate total length for a particular patient.

The preparation of the patient's ankle includes, in summary, preparing an ankle joint space by resecting the trochlea portion of the talar bone and the distal end of the tibia. The total ankle implant system 100 of the present disclosure is for a patient whose deterioration of the talar bone is minimal such that only minimal amount of the trochlea portion of the talar bone needs to be removed and requires a chamfered cut resection of the trochlea portion. The chamfered cut involves making a shallow flat resection of the superior (top) portion of the trochlea and making chamfer cuts in the anterior portion and the posterior portion of the trochlea. The resulting ankle joint space is significantly shorter in the proximal-distal direction than in patients whose talar bone required removal of a substantial amount of the trochlea portion via a flat cut resection.

The talar dome implant 230 for the total ankle implant system 100 of the present disclosure accordingly has the reverse-chamfered bone contacting distal surface 253 described below with reference to FIG. 4.

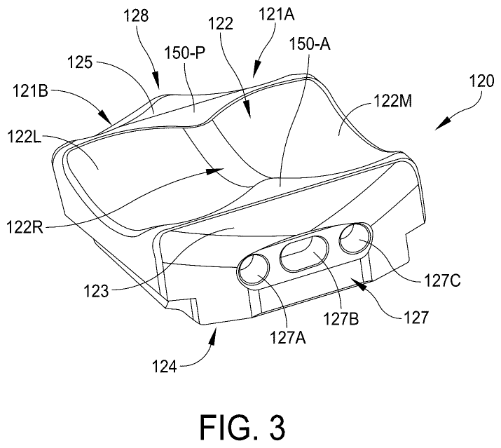

FIG. 3 shows a polymer insert 120 of the total ankle arthroplasty implant 100 according to an embodiment of the present disclosure. The polymer insert 120, as described herein, is configured to be inserted into the tibia tray 110. The polymer insert 120 comprises a concave distal surface 122, a proximal face 124 on the opposite side thereof, an anterior end 127, and a posterior end 128.

The concave distal surface 122 is provided between a posterior ridge 150-P and an anterior ridge 150-A. The concave distal surface 122 comprises two generally symmetrical portions: a lateral concave distal surface 122L and a medial concave distal surface 122M. To seat the polymer insert 120 into the tibia tray 110, the polymer insert 120 is squeezed between the tibia tray 110 and the talar dome implant 230. The posterior face 125 of the polymer insert 120 facing the talar dome implant 230 is configured to facilitates the polymer insert to slide against the articulating surface of the talar dome implant 230 and allows the polymer insert 120 to squeeze between the tibia tray 110 and the talar dome implant 230. Once the talar dome implant 230 is past the posterior ridge 150-P, the talar dome implant 230 comes to rest in the recesses of the concave distal surface 122 between the posterior ridge 150-P and the anterior face 123. The proximal face 124 of the polymer insert is configured to slidingly engage the tibia tray 110.

Between the lateral concave distal surface 122L and the medial concave distal surface 122M includes a ridge 122R configured and arranged to engage with the talar dome implant's sulcus or trough in its articulating surface. The concave distal surface 122 is non-abrasive and configured to protect the smooth articular surface of the talar dome implant 130.

The concave distal surface 122 are generally complementary to the dome of the talar dome implant 230, allowing for optimal post-operative ankle movement.

FIG. 4 shows a talar dome implant 230 with a chamfered profile according to an embodiment of the present disclosure. A surgical clamp may be used by a surgeon to insert a the talar dome implant 230 into or remove a talar dome implant 230 from a joint space during an ankle joint prosthetic procedure. The proximal surface of the talar dome implant 230 can be smooth and articular. The talar dome implant 230 includes a pair of convex proximal articular surfaces 250M, 250L (250M being on the medial side and 250L being on the lateral side), an anterior side 251, a posterior side 252, and a bone-contacting distal surface 253 opposite from the convex proximal articular surfaces 250M, 250L.

The bone-contacting distal surface 253 of the talar dome implant 230 can be described as a concave surface or a reverse-chamfered surface to engage the dome of a talar bone that has been resected with chamfer cuts. The bone-contacting distal surface 253 is configured to abut against a resected surface of the talus of the ankle joint having a shape that is complementary to the bone-contacting distal surface 253.

Alternatively or additionally, the bone-contacting distal surface 253 includes an anterior sloped surface 255, a central plantar surface 256, and a posterior sloped surface 257. The anterior end of the bone-contacting distal surface 253 has an anterior edge 254. The posterior end of the bone-contacting distal surface 253 has a posterior edge 258. The anterior sloped surface 255 is inclined upwardly from the anterior edge 254 to the central plantar surface 256, and the posterior sloped surface 257 is inclined downwardly from the central plantar surface 256 to the posterior edge 258.

In some examples, the talar dome implant 230 can further include one or more pegs 231 rigidly protruding from the anterior sloped surface 255, wherein the one or more pegs 231 are tapered for embedment into the talus of the ankle joint, and configured to be impacted into the talus of the ankle joint.

The reverse-chamfered design of the talar dome implant 230 results in a larger surface area to make contact between the talar dome implant 230 and the talar bone. Additionally, as the connecting face is angled due to the reverse-chamfering shape, the connection between the talar bone is improved when shear forces are experienced. A chamfer cut into the talar bone generally results in cutting away less bone material, leaving behind a larger volume of healthy talar bone. Additionally, the reverse-chamfered design requires a smaller surgical opening to fit the smaller-profiled implant into position. Therefore, the chamfered talar dome implant 230 when used in conjunction with the multi-component stem 105 can allow for a materially stronger connection to both the talar bone as well as the tibia bone in a total arthroplasty while maintaining a small surgical opening profile.

Generally, a surgeon holds talar dome implant 230 of the total ankle arthroplasty implant 100 and inserts it into a joint space during an ankle joint arthroplasty procedure. Once the talar dome implant 230 is in position in the joint space, generally the surgeon impacts the talar dome implant 230 into the prepared talus. In some embodiments, a first and second recess in the talar dome implant 230 are configured to connect to protrusions from a holding apparatus which enables the surgeon to securely grasp the talar dome implant 230 when preparing to insert the talar dome implant 230 into the joint space. The surgeon can then easily and securely insert the talar dome implant 230 into the joint space and subsequently impact the talar dome implant 230 into the prepared talus by applying an impact force to the proximal end of the holding apparatus or applying an impact force to the proximal end of an additional impaction tool that engages the proximal end of the talus.

FIG. 5A shows an anterior-side view of the multi-component stem 105 and a tibia tray 110 coupled together according to an embodiment of the present disclosure. FIG. 5B shows an section view of the tibia tray shown in FIG. 5A. The tibia tray 110 comprises an anterior end configured to receive an installation instrument for holding the tibia tray 110, a posterior end, a top surface including the multi-component stem 105, configured for engaging a distal end of a tibia bone, and a bottom surface configured for engaging a polymer insert 120 (not shown) of total ankle arthroplasty implant 100.

The tibia tray 110 is configured with a slot 141 that opens to the anterior end 140 of the tibia tray for receiving hooks of an installation instrument to form a secure engagement. The slot 141 has side pockets 142M, 142L on the medial and lateral sides of the slot 141. The side pockets 142M, 142L receive and engage the hooks so that when two jaws of the installation instrument are expanded in the medial-lateral direction, the hooks catch the side pockets 142M, 142L and prevent the jaws from being pulled out of the slot 141.

FIG. 2A shows a top stem 106 of the multi-component stem 105 of the total ankle arthroplasty implant 100 according to an embodiment of the present disclosure. FIG. 2B shows a mid stem 107 of the multi-component stem 105 of the total ankle arthroplasty implant 100 according to an embodiment of the present disclosure. FIG. 2A shows a base stem 108 of the multi-component stem 105 of the total ankle arthroplasty implant 100 according to an embodiment of the present disclosure.

The top stem 106 includes a tapered proximal end 106p that has a conical shape to facilitate being driven into the intramedullary tissue, while doing minimal damage to that intramedullary tissue. Preferably, a path may be drilled or reamed into the intramedullary tissue before the top stem 106 is installed. The top stem 106 also includes ridges and convex surfaces to facilitate affixing the top stem 106 and consequently the multi-component stem 105 into the tibia. The top stem 106 also includes a distal end 106d that is adapted to connect to a mid stem 107, a base stem 108, or any combination thereof. The distal end 106d of the top stem 106 can be threaded or otherwise connected into a lower component.

The mid stem 107 is configured to connect at its proximate end 107p to a top stem 106, another mid stem 107, or any combination thereof. The mid stem 107 comprises a distal end 107d that is configured to connect to another mid stem 107, a base stem 108, or any combination thereof. The mid stem 107 also comprises a proximate end 107p that can be provided with a male-type thread to make a threaded or other connection with the top stem 106 or another mid stem 107. The distal end 106d of the top stem 106 is configured with a complementary female threaded recess or other connection to establish the threaded or other engagement.

The base stem 108 is configured to connect at its proximate end 108p to a top stem 106, a mid stem 107, or any combination thereof. The base stem 108 comprises a distal end 108d that is configured to connect to the tibia tray 110. The proximate end 108p can be threaded to make a threaded or other connection with the top stem 106, or a mid stem 107. The distal end 107d of the mid stem 107 is configured with a complementary female threaded or other connection recess to establish the engagement.

The distal end 108d of the base stem 108 is configured to engage with the tibia tray implant's proximal surface 145.

The proximal end 108p of the base stem 108 can include a threaded body or other connection feature. The distal end 106d of the top stem 106 can include a threaded hole configured to accept the threaded body. The proximal surface 145 of the tibia tray implant 110 can include a male-type friction-fit taper such as a Morse taper, and the distal end 108d of the base stem 108 can include a tapered socket configured to accept the male-type friction-fit taper.

The base stem 108 may be slightly larger in diameter than the mid stem 107. Being slightly larger may function to provide more snug fit with the bone. Being slightly larger may function to “plug” the hole in the existing bony wall, reducing risk of infection and improving overall acceptance of the total ankle arthroplasty implant 100.

After the ankle joint space is prepared, an intramedullary cavity is reamed into the distal end of the tibia to receive the multi-component modular stem 105. As the multi-component stem 105 comprises multiple stem components 106, 107, and 108, the stem components are, individually or in pre-assembled arrangement, inserted into the ankle joint space then inserted into the intramedullary cavity.

The insertion of the modular stem components may involve the following steps. For the cases where the patient only requires the shortest assembly of the multi-component stem 105 that includes one top stem 106 and a base stem 108, the top stem 106 inserted into the ankle joint space with the tapered proximal end 106p of the top stem 106 entering the ankle joint space first. As the proximal end 106p is tapered, the top stem 106 can be rotated so that the tapered proximal end 106p is pointing into the intramedullary cavity of the prepared tibia.

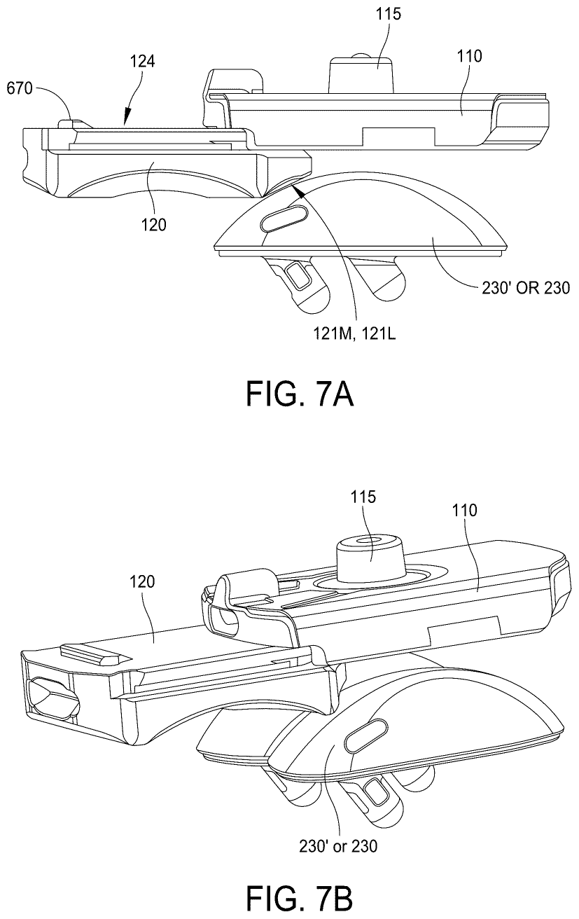

Next, the base stem 108 is inserted into the ankle joint space and threaded onto the top stem 106 that is positioned in the intramedullary cavity using an appropriate wrench. Then, the tibia tray component 110 is inserted into the ankle joint space and the base stem distal end 108d of the base stem 108 is attached to the proximal end of the tibia tray component 110 by impacting the tibia tray component's male tapered feature 115 into the base stem 108 using an appropriate wrench, tray holding tool, and strike rod. The male tapered feature 115 of the tibia tray component 110 can be seen is FIGS. 7A and 7B.

In another embodiment, the top stem 106 and the base stem 108 can be first assembled together into a mini-assembly. The mini-assembly can be formed by threading the proximate end 108p of the base stem 108 into the distal end 106d of the top stem 106. This mini-assembly is then inserted into the ankle joint space with the tapered proximal end 106p of the top stem 106 entering the ankle joint space first. As the proximal end 106p is tapered, the mini-assembly can be rotated so that the tapered proximal end 106p of the top stem 106 is pointing into the intramedullary cavity of the prepared tibia. According to the present disclosure, the bases stem 108 has a height 108h (see FIG. 2N) that is sufficiently small so that this maneuver can be made within the small ankle joint space. With the existing multi-component modular stem systems, the corresponding stem components are too tall and this maneuver is not possible where the patient's talar bone is resected with chamfer cuts.

After the mini-assembly of the top stem 106 and the base stem 108 is inserted into the intramedullary canal, the tibia tray component 110 is inserted into the ankle joint space and the Morris taper connection is made by impacting the male taper portion of the tibia tray component into the female Morris taper at the base end 108d of the base stem 108.

For the cases where the patient requires longer assembly of the multi-component stem 105, a mini-assembly of one top stem 106 and a mid stem 107 is first assembled by threading the proximate end 107p of the mid stem 107 into the distal end 106d of the top stem 106. This mini-assembly is then inserted into the ankle joint space with the tapered proximal end 106p of the top stem 106 entering the ankle joint space first. As the proximal end 106p is tapered, the mini-assembly can be rotated so that the tapered proximal end 106p of the top stem 106 is pointing into the intramedullary cavity of the prepared tibia. According to the present disclosure, the mid stem 107 has a height 107h (see FIG. 2I) that is sufficiently small so that this maneuver can be made within the small ankle joint space. With the existing multi-component modular stem systems, the corresponding stem components are too tall and this maneuver is not possible where the patient's talar bone is resected with chamfer cuts.

After the mini-assembly of the top stem 106 and mid stem 107 is inserted into the intramedullary cavity, a second mid stem 107 is inserted into the ankle joint space and attached to the mini-assembly. The attachment is achieved by threading the proximal end 107p of the second mid stem 107 into the distal end 107d of the first mid stem 107. This procedure is repeated for any additional mid stems that may be required to achieve the total desired length for the multi-component stem 105 assembly. Then, a base stem 108 is inserted into the ankle joint space and threaded onto the last mid stem 107 component in the multi-component stem 105 that has been assembled in the intramedullary cavity. Then, the tibia tray component 110 is inserted into the ankle joint space and a Morris taper connection is made by impacting the male taper portion of the tibia tray component into the female Morris taper at the base end 108d of the base stem 108.

After the multi-component stem 105 and the tibia tray component 110 are positioned in place, the talar dome implant 230 can be affixed to the prepared talar bone. Then, the polymer insert 120 can be placed in position by wedging it between the tibia tray component 110 and the talar dome implant 230 and sliding the polymer insert 120 into the tibia tray component 110.

Once the tibia tray 110 is attached to the end of installation instrument, the polymer insert 120 is positioned in place to be inserted into the tibia tray 110. To hold the polymer insert 120 for the insertion procedure, the installation instrument comprises two interference connection pins, and the polymer insert implant 120 has corresponding two holes 127A, 127C that receive the connection pins to establish interference-fit engagement. The connection pins are part of the installation instrument and extend out from the installation instrument in the posterior direction. The connection pins are designed to be compressible and the corresponding holes 127A, 127C are sized such that the connection pins fit tightly.

The tibia tray 110 comprises a recess 147 on the inferior or distal side 146 for receiving and engaging the polymer insert 120. The recess 147 is configured with side rail structures 148A-B that enables the tibia tray 110 and the polymer insert 120 to slide into the recess 147 and allow the two components 110, 120 to engage and hold together.

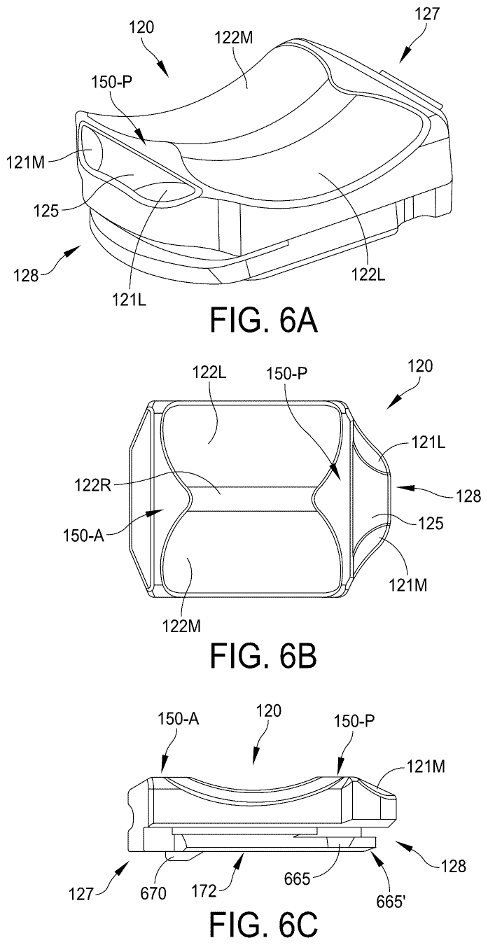

Referring to FIGS. 3, 6A, 6B, and 6C, an embodiment of the polymer insert 120 comprising a novel feature according to an aspect of the present disclosure is now described. FIG. 6A shows an isometric view of a polymer insert 120. FIG. 6B shows a proximal-side view of a polymer insert 120. FIG. 6C shows an inferior-side view of a polymer insert 120.

The posterior face 125 of the polymer insert 120 can be configured to include two dish features 121M, 121L to facilitate inserting the polymer insert between the tibia tray implant 110 and the talar dome implant 230 while engaging the polymer insert with the tibia tray's recess 147. The dish feature 121M is on the medial side of the polymer insert 120 and the dish feature 121L is on the lateral side of the polymer insert 120. The dish features 121M, 121L can be implemented in multiple versions, e.g., as compound angled faces, concave dishes, multifaceted ramps, or a combination thereof. In the example embodiment of the dish features 121M, 121L described herein, the dish features 121M, 121L have concave surfaces and thus can be described as scalloped or cove cut portions.

Inserting the polymer insert 120 into the installed tibia tray implant 110 by wedging the polymer insert 120 between the tibia tray implant 110 and the talar dome implant 230 can be a challenge, because muscles and ligaments hold the patient's foot and the tibia bone together at the ankle, and the ankle has limited ability to plantar flex the foot to open the ankle joint. As the polymer insert 120 is being inserted into the tibia tray's recess 147 from the anterior side of the ankle joint in an anterior-to-posterior direction, the polymer insert 120 is being wedged between the tibia tray implant 110 and the articulating surfaces of the talar dome 230. This generally requires forcefully pushing the polymer insert 120 between the tibia tray implant 110 and the talar dome 230 to temporarily pry apart patient's foot and the tibia and allow the polymer insert to ride up and over the articulating surfaces of the talar dome implant 230 as the polymer insert 120 is slid along the side rail structures of the tibia tray implant 110.

Each of the dish features 121M, 121L has a concave contour that is oriented at an angle such that the surface of each of the dish features 121M, 121L is at an incline starting from the posterior end 128, which is the leading end when being inserted, of the polymer insert 120 to the posterior ridge 150-P. This configuration of the dish features 121M, 121L provides ramps for the corresponding articulating surfaces 250M, 250L of the talar dome implant 230 to interact with as the polymer insert 120 is wedged into position between the tibia tray implant 110 and the talar dome implant 230.

Because a pair of the dish features 121M and 121L are provided that correspond with the pair of articulating surfaces 250M, 250L of the talar dome 230, the dish features 121M, 121L also helps keep the polymer insert 120 and the talar dome 230 aligned as the polymer insert 120 is being wedged into place. The convex surfaces of the dish features 121M, 121L are configured to complement the curvature of the articulating surfaces 250M, 250L of the talar dome implant 230 which help the polymer insert 120 maintain its alignment with the talar dome implant 230 and the tibia tray implant 110 as the polymer insert 120 is pushed into the space between the tibia tray and the talar dome in the Anterior to Posterior (A-P) direction along the side rail structures of the tibia tray. This prevents the polymer insert 120 from wiggling or sliding out of alignment as the polymer insert is pushed up and over the articular surfaces of the talar dome implant 230, ultimately casing the polymer insert 120 insertion process.

The portion of the polymer insert 120 that forms the posterior face 125 between the dish features 121M, 121L form a positive keel feature, complementary to the recess 250R down the center of the talar dome implant 230. This keel feature takes advantage of the recess in the talar dome implant 230, facilitating alignment between the polymer insert 120 and the talar dome implant 230. The dish features 121M, 121L, because of their curvature complementing the talar dome implant 230, also make the foot angle less important during installation-meaning the foot does not need to be maximally plantarly flexed in order for the polymer insert 120 to be installed in the implant 100.

FIG. 7A shows an inferior-side view of the polymer insert 120 being inserted between the tibia tray component 110 and a talar dome to show the engagement of the dish features 121M, 121L with a talar dome during the insertion. The particular talar dome implant shown in FIG. 7A is a flat-cut talar dome 230′ but different types of talar dome implant could be in use depending on the condition of the patient's talus bone. For example, the polymer insert 120 can also be used with the reverse-chamfered talar dome 230 described above. FIG. 7B shows a detailed isometric view of the components shown in FIG. 7A. In some embodiments, some portions of the bone contacting surfaces of the talar dome implants and/or the tibia tray implant can be configured as porous surfaces that can facilitate bone in-growth.

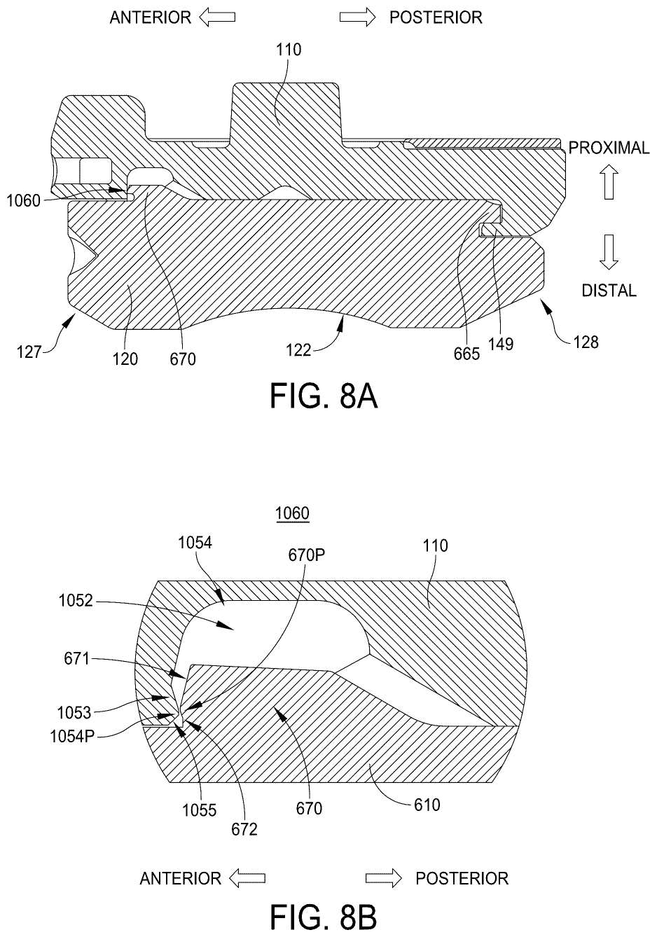

According to some embodiments, FIGS. 5A, 5B, 8A and 8B depict an ankle arthroplasty implant system including a tray implant 110 configured for engaging a distal end of a tibia in a patient's ankle joint. The tray implant 110 includes the distal side 146, where the distal side 146 includes a main recess 147. The ankle arthroplasty implant system also includes a polymer insert 120, configured to be inserted in the tibia tray implant's main recess 147. The main recess 147 is open toward the distal direction and also open at the anterior end of the tray implant 110.

The polymer insert 120 is received into the main recess 147 from the anterior end of the tray implant 110 and the main recess 147 comprises side rail structures 148A, 148B that are configured to guide the polymer insert 120 as the polymer insert 120 is inserted into the main recess 147.

Referring to FIG. 6C, the polymer insert 120 may further include a retaining rail structure 665 configured to engage with the side rail structures 148A, 148B and guide the polymer insert 120 when the polymer insert 120 is being inserted into the main recess 147. The retaining rail structure 665 extend along the medial and lateral sides of the polymer insert 120 as well as the posterior end 128 of the polymer insert 120. The protruding profile 665′ of the retaining rail structure 665 at the posterior end of the polymer insert 120 can be seen in FIG. 6C.

Referring to FIGS. 5A and 8A, the main recess 147 is closed at the posterior end of the tray implant 110. FIG. 8A is a sectional view of an assembly of the tray implant 110 and the polymer insert 120, where the section is taken along a plane that extends through the assembly in anterior-posterior (A-P) direction. The closed posterior end of the main recess 147 comprises a ledge 149 that is configured to engage and cooperate with the protruding profile 665′ of the retaining rail structure 665 at the posterior end of the polymer insert 120 to securely hold the posterior end of the polymer insert 120 against the tray implant 110 when the polymer insert 120 is fully inserted into the tray implant 110. This engagement between the ledge 149 and the rail structure 665 can be seen in FIG. 8A.

FIG. 8A shows a cross-section view of an assembly of the tibia tray implant 110 and the polymer insert 120 after the polymer insert 120 is fully inserted into the tibia tray implant 110, where the attachment between the tibia tray implant 110 and the polymer insert 120 is enhanced by a new locking feature 1060. FIG. 8B shows a detailed area diagram of the additional locking mechanism 1060 noted in FIG. 8A.

As the polymer insert 120 is advanced in anterior to posterior direction along the side rails 148A, 148B of the tibia tray 110, upon reaching the posterior end of the tibia tray 110, the polymer insert 120 is configured to lock in place via operation of the locking feature 1060.

Referring to FIGS. 8A-B, according to some embodiments, the polymer insert 120 and the tibia tray implant 110 are configured to provide the additional locking mechanism 1060 near the anterior ends of the tray implant 110 and the polymer insert 120. The additional locking mechanism 1060 is formed by a locking tab 670 provided on the proximal surface 124 of the polymer insert 120 and a locking recess 1052 provided on the distal side 146 of the tray implant 110 within the main recess 147. The locking recess 1052 is defined by an interior wall portion 1054 of the main recess 147. The locking tab 670 is positioned on the proximal surface 124 of the polymer insert 120 so that when the polymer insert 120 is fully slid into the main recess 147, the locking tab 670 is received in the locking recess 1052 as shown in FIG. 8A.

Referring to FIG. 8B, the locking tab 670 comprises a first protrusion 670P and the interior wall 1054 comprises a second protrusion 1054P, and the additional locking mechanism 1060 comprises the first protrusion 670P and the second protrusion 1054P. The first protrusion 670P extends anteriorly from the locking tab 670 and the second protrusion 1054P extends posteriorly from the interior wall 1054. Thus, the two protrusions extend in opposing directions. The positions of the locking tab 670 and the locking recess 1052 with respect to each other are such that as the locking tab 670 is seated within the locking recess 1052, the first protrusion 670P and the second protrusion 1054P make contact and physically interfere to form an interference fit that is sufficiently intimate to form a mechanical locking engagement. The moment at which the two protrusions make contact will be referred to herein as the contact point.

According to some embodiments, the first protrusion 670P and the second protrusion 1054P are misaligned in the proximal-distal direction by a predetermined amount so that as the locking tab 670 is being seated within the locking recess 1052, the first protrusion 670P and the second protrusion 1054P will make an initial contact and then move past the initial contact point by a predetermined desired amount and thus forming the interference fit.

In some embodiments, the first and second protrusions each has a tip that is at a maximum protrusion, and the misalignment is such that the tip of the first protrusion 670P is positioned further in proximal direction than the tip of the second protrusion 1054P. In some embodiments, the misalignment is such that the tip of the second protrusion 1054P is positioned further in proximal direction than the tip of the first protrusion 670P.

In some embodiments, the first protrusion 670P is defined by a pair of back-angled surfaces 671 and 672. The second protrusion 1054P is defined by another pair of back-angled surfaces 1053 and 1055.

Unless otherwise expressly stated, it is in no way intended that any method set forth herein be construed as requiring that its steps be performed in a specific order, nor that with any apparatus, specific orientations be required, unless specified as such. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including matters of logic with respect to arrangement of steps, operational flow, order of components, or orientation of components; plain meaning derived from grammatical organization or punctuation, and; the number or type of embodiments described in the specification.

The disclosed devices and systems may be used in a wide variety of surgical methods and procedures. The disclosed devices and systems advantageously enable orthopedic surgical procedures (e.g., ankle joint prosthetic procedures).

What is claimed is:

1. An ankle arthroplasty implant system, comprising:
a multi-component tibia stem configured for engaging a distal end of a tibia in a patient's ankle joint and including a first component stem and a second component stem;
a tray implant, including:
an anterior side, including a slot extending into the tray implant, the slot generally perpendicular to the anterior side,
one or more side pockets located on the medial and/or lateral sides of the slot, extending into the tray implant and generally parallel to the slot,
a proximal surface configured to engage the multi-component stem, and
a distal side, including a recess;
a talar dome implant,
including:
a convex proximal articular surface,
an anterior side,
a posterior side, and
a bone-contacting distal surface opposite from the convex proximal articular surface, wherein the bone-contacting distal surface is a concave surface;
wherein the bone-contacting distal surface is configured to abut against a resected surface of the talus of the ankle joint having a shape that is complementary to the bone-contacting distal surface; and
a polymer insert configured to be inserted in the tray implant recess, and including:
an anterior face, including one or more blind holes;
a concave distal surface configured to substantially conform to and engage with the proximal articular surface of the talar dome implant.

2. The ankle arthroplasty implant system of claim 1, wherein the polymer insert includes a polymer insert posterior face, and the polymer insert posterior face includes a medial dish on a medial insert side of the polymer insert posterior face, and a lateral dish on a lateral insert side of the polymer insert posterior face, the medial dish and the lateral dish configured to ease wedging the polymer insert between the tray implant recess.

3. The ankle arthroplasty implant system of claim 1, wherein the recess of the tray implant includes side rail structures and a locking mechanism, the side rail structures configured to guide the polymer insert into the recess, engage and hold the tray implant and the polymer insert together via the side rail structures, the locking mechanism, and a complementary locking mechanism of the polymer insert.

4. The ankle arthroplasty implant system of claim 3, wherein: the locking mechanism includes a back-angled face; and the complementary locking mechanism includes a back-angled face.

5. The ankle arthroplasty implant system of claim 1, wherein the talar dome implant further comprises one or more pegs rigidly protruding from the anterior sloped surface, wherein the one or more pegs are configured for embedment into the talus of the ankle joint and configured to be impacted into the talus of the ankle joint.

6. The ankle arthroplasty implant system of claim 1, wherein:
the second component stem comprises a proximal end and a distal end, wherein the distal end is configured to engage with the tray implant's proximal surface; and
the first component stem comprises a proximal end and a distal end, wherein the distal end of the first component stem is configured to engage with the proximal end of the second component stem.

7. The ankle arthroplasty implant system of claim 6, wherein:
the proximal end of the second component stem includes a threaded body;
the distal end of the first component stem includes a threaded hole configured to accept the threaded body;
the proximal surface of the tray implant includes a friction-fitting taper; and
the distal end of the second component includes a taper socket configured to accept the friction-fitting taper.

8. The ankle arthroplasty implant system of claim 1, wherein:
the first component stem is a top stem and includes a top-type distal end;
the second component stem is of a base-type component stem and includes a base-type proximal end and a base-type distal end;
the first component stem is configured to only engage via coupling with a base-type proximal end of the second component stem or a mid-type proximal end of a mid-type component stem of the multi-component tibia stem via the top-type distal end;
the second component is configured to only engage via coupling with:
a top-type distal end of the first component stem or a mid-type distal end of the mid-type component stem via the base-type proximal end, and
the tray implant proximal surface via the base-type distal end.

9. The ankle arthroplasty implant system of claim 8, further comprising:
a third component stem of the multi-component tibia stem;
wherein:
the third component stem is of the mid-type component stem, and includes a third component proximal face and a third type distal face;
the third component stem is configured to only engage via coupling with a top-type distal face or mid-type distal face via the third component proximal face; and
the third component stem is configured to only engage via coupling with a mid-type proximal face or a base-type proximal face via the third component distal face.

10. The ankle arthroplasty implant system of claim 1, wherein:
the multi-component tibia stem engages with the distal end of the tibia in the patient's ankle joint via the first component stem and the second component stem, the first component stem tapered for embedment into the tibia and configured to be inserted into a drilled intramedullary path in the tibia; and
the tray implant is further configured to engage with the distal end of the tibia via bone cement placed on the tray implant proximal surface.

11. The ankle arthroplasty implant system of claim 1, wherein the bone-contacting distal surface comprises an anterior sloped surface and a posterior sloped surface.

12. The ankle arthroplasty implant system of claim 11, wherein the bone-contacting distal surface further comprises a central planar surface.

13. The ankle arthroplasty implant system of claim 1, wherein the concave surface of the bone-contacting distal surface is a curved surface.

14. A polymer insert for forming an interface between a tibia tray component and a talar component of an ankle prosthesis, the polymer insert comprising:
a concave distal surface configured for articulatingly engaging articulating surface of the talar component;
a posterior face having a medial side and a lateral side and including:
a medial dish portion on the medial side of the posterior face,
a lateral dish portion on the lateral side of the posterior face; and
a posterior ridge separating the concave distal surface from the posterior face, wherein the medial dish portion and the lateral dish portion are oriented and contoured to facilitate the polymer insert being inserted between the tibia tray component and the talar component by aligning the polymer insert and reducing insertion force required.

15. The polymer insert of claim 14, wherein the medial dish portion and the lateral dish portion have concave surfaces.

16. The polymer insert of claim 15, wherein each of the medial dish portion and the lateral dish portion has a convex contour that is oriented at an angle such that the surface of each of the dish portions is at an incline starting from the posterior end of the polymer insert to the posterior ridge.

17. A method for forming an intramedullary path in a patient's tibia through an associated calcaneus bone comprising:
drilling through a calcaneus;
forming an ankle joint space by resecting a distal end of a patient's tibia and the proximal end of the patient's talar bone so as to form an opening into the ankle joint space;
forming a convex cut to the resected proximal end of the patient's talar bone;
reaming the intramedullary path in the tibia through the calcaneus and talar bones to a predetermined diameter and predetermined depth;
inserting a first component stem of a multi-component stem into the ankle joint space and into the intramedullary path;
inserting a second component stem of the multi-component stem into the ankle joint space; and
coupling the second component stem to the first component stem so as to form an assembled stem.

18. The method of claim 17 wherein the second component stem comprises a proximal end and a distal end, wherein the distal end is configured to engage with a tray implant's proximal surface; and
the first component stem comprises a proximal end and a distal end, wherein the distal end of the first component stem is configured to engage with the proximal end of the second component stem.

19. The method of claim 17 wherein the first component stem is a top stem and includes a top-type distal end, the second component stem is of a base-type component stem and includes a base-type proximal end and a base-type distal end, the first component stem is configured to only engage via coupling with a base-type proximal end of the second component stem or a mid-type proximal end of a mid-type component stem of the multi-component tibia stem via the top-type distal end, the second component is configured to only engage via coupling with a top-type distal end of the first component stem or a mid-type distal end of the mid-type component stem via the base-type proximal end and a tray implant proximal surface via the base-type distal end.

20. The method of claim 17 wherein a bone-contacting distal surface comprises an anterior sloped surface and a posterior sloped surface, the bone-contacting distal surface further comprises a central planar surface, and a concave surface of the bone-contacting distal surface is a curved surface.
